(19) United States
(12) Patent Application Publication
(10) Pub. No.: US 2016/0287178 A1
(43) Pub. Date: Oct. 6, 2016

(54) SENSOR GUIDE WIRE HAVING A PROXIMAL TUBE WITH IMPROVED TORQUE PERFORMANCE AND MAINTAINED LOW BENDING STIFFNESS

(71) Applicant: ST. JUDE MEDICAL COORDINATION CENTER BVBA, Zaventem (BE)

(72) Inventors: Sathees RANGANATHAN, Sundbyberg (SE); Anna NORLIN-WEISSENRIEDER, Lidingö (SE)

(73) Assignee: ST. JUDE MEDICAL COORDINATION CENTER BVBA, Zaventem (BE)

(21) Appl. No.: 15/083,615

(22) Filed: Mar. 29, 2016

Related U.S. Application Data
(60) Provisional application No. 62/140,132, filed on Mar. 30, 2015.

(51) Int. Cl. A61B 5/00

(52) U.S. Cl. CPC A61B 5/6851; A61M 2025/09066

(57) ABSTRACT

A sensor guide wire for an intravascular measurement of a physiological variable in a living body includes a sensor element configured to measure the physiological variable, and a proximal tube comprising a first material having a first Young's modulus and a second material having a second Young's modulus. The second Young's modulus is higher than the first Young's modulus. The second material is configured to improve torqueability.

### CROSS-REFERENCE TO RELATED APPLICATION

The present application claims the benefit of U.S. Provisional Application No. 62/140,132, filed on Mar. 30, 2015, which is hereby incorporated by reference in its entirety.

### BACKGROUND

The invention generally relates to the area of medical devices. More particularly, the present invention concerns a sensor guide wire for intravascular measurements of a physiological or other variable, e. g. pressure or temperature, inside a living human or animal body, the sensor guide wire including a proximal tube formed of two or more materials, each having a different Young's modulus to improve torque performance and maintain a low bending stiffness.

Equipment and processes have been developed for assisting medical personnel, such as physicians, in diagnosing physiological conditions of a patient. For example, sensor guide wires in which a sensor is mounted at the distal end thereof have been developed. The sensor may be, for example, an intra-vascular pressure sensor that is arranged to measure blood pressure at various points within the vasculature to facilitate locating and determining the severity of, for example, steno sis or other disruptors of blood flow within the vessels of the living body.

Sensor and guide wire assemblies in which a sensor is mounted at the distal end of a guide wire are known. In U.S. Pat. No. Re. 35,648, which is assigned to the present assignee, an example of such a sensor guide wire is disclosed, where a sensor guide wire comprises a sensor element, an electronic unit, at least one signal transmitting cable connecting the sensor element to the electronic unit, a flexible tube having the cable disposed therein, a solid metal wire, and a coil attached to the distal end of the solid wire. The sensor element comprises a pressure sensitive device, e.g. a membrane, with piezoresistive elements connected in a Wheatstone bridge-type of arrangement mounted thereon.

The above-mentioned solid metal wire, also called the core wire, extends from the distal end of the sensor guide wire to the proximal portion, where a male connector is arranged, and determines in part the overall mechanical properties, such as flexibility, torqueability and pushability, of the sensor guide wire. Sensor and guide wire assemblies for intravascular measurements are generally long, e.g. 100-300 cm, and have a small diameter, e.g. 0.35 mm. The core wire often extends along essentially the entire length of the sensor guide wire.

A proximal tube may extend from a proximal male connector to a jacket, inside which a sensor element is arranged. As an alternative, a proximal tube may extend from a proximal male connector to a coil, which, in turn, is connected to such a jacket. The core wire is inserted through a lumen of the proximal tube. The core wire may be longer than the proximal tube, and may extend from the proximal male connector, through the jacket, and to the distal tip of the sensor guide wire.

A core wire is a wire typically made out of metal and is typically of complex mechanical construction since it has to be steered often several feet into a patient, for example, from an opening in the femoral artery in the leg of the patient up to the heart through tortuous blood vessels. The mechanical characteristics (such as maneuverability, steerability, torqueability, and pushability) of a guide wire are very important to a surgeon because the surgeon grasps the proximal end of a guide wire (sticking outside the patient), and by manipulating the proximal end, steers the distal end of the guide wire, which is often several feet away.

Maneuverability describes the overall ability of the guide wire to travel through complex anatomies and is influenced by a number of factors including flexibility, strength, torqueability, pushability and friction within the anatomical environment.

Steerability describes a guide wire's ability to react to torque and push so that the distal end reaches parts of vessels as intended by the user. Steerability is primarily determined by the guide wire's stiffness and its thickness or strength.

Torqueability describes the ability of the guide wire to transmit a rotational displacement along the length of the sensor guide wire. When the rotational movements by the physician translate exactly to the tip of the sensor guide wire within the anatomy, the torque performance is high, so called “1:1” torque ratio.

Pushability describes the ability of the guide wire to transmit a longitudinal force from the proximal end of the shaft to the distal end. When a guide wire shaft has been designed to optimize pushability, it is easier for the physician to maneuver the sensor guide wire to the desired spot.

The guide wire is steered through the arteries, rather than being “pushed” or simply “introduced” through the arteries. A typical guide wire is very thin (typically 0.35 mm or less in diameter). Since the artery wall is soft, any attempt to use the artery itself as a guide for the guide wire could lead to penetration of the artery wall. The guide wire must be steered, for example, from an opening in the femoral artery in the leg of the patient up to the heart through tortuous blood vessels.

### SUMMARY

In order to increase torqueability, it is known to increase the bending stiffness of the proximal tube by selecting a material with a high Young's modulus. However, as the bending stiffness increases, the proximal tube exerts more pressure on the artery/catheter walls, thereby increasing the friction force on the guide wire. The increased friction contributes to a reduction in torque, which is counteractive to the purpose of the design. In addition, a higher bending stiffness increases the risk of the guide wire penetrating a blood vessel.

Thus, there is a need for an improved sensor guide wire having a proximal tube that exhibits improved torque performance, while maintaining a low bending stiffness.

In one embodiment, a sensor guide wire for an intravascular measurement of a physiological variable in a living body includes a sensor element configured to measure the physiological variable, and a proximal tube comprising a first material having a first Young's modulus and a second material having a second Young's modulus. The second Young's modulus is higher than the first Young's modulus. The second material is configured to improve torqueability.

In another embodiment, a method of forming a proximal tube for a sensor guide wire for an intravascular measurement of a physiological variable in a living body is described. The method includes providing at least one sheet made of one of 1) a first material having a first Young's modulus or 2) a second material having a second Young's modulus, providing a plurality of strips made of the other of the first material and the second material, joining the first material and the second material, and forming the joined first material and the second material into a hollow tube having the plurality of strips spaced along a length of the hollow tube. The second Young's modulus is higher than the first Young's modulus.

In yet another embodiment, a guide wire includes a tubular member having along its length a first material having a first Young's modulus and a second material having a second Young's modulus. The second Young's modulus is higher than the first Young's modulus. The tubular member has alternating portions of the first material and the second material.

All references cited in this disclosure are hereby incorporated by reference in their entireties for the devices, techniques, and methods described therein relating to medical sensors and devices, and for any disclosure relating to medical sensors and devices.

### BRIEF DESCRIPTION OF THE DRAWINGS

The features, aspects and advantages of the present invention will become apparent from the following description, appended claims, and the accompanying exemplary embodiments shown in the drawings, which are briefly described below.

FIG. 1 is a schematic drawing showing a system for intravascular measurement according to one embodiment of the present invention.

FIG. 2 is a cross-sectional view of a sensor guide wire used in the system of FIG. 1 according to one embodiment of the present invention.

FIG. 3 is a schematic drawing showing a first embodiment of a proximal tube (prior to formation of the hollow tube) formed of a sheet of a first material having a first Young's modulus and a plurality of strips of a second material having a second Young's modulus higher than the first Young's modulus.

FIG. 4 is a schematic drawing showing the proximal tube according to the embodiment of FIG. 3, after formation of the hollow tube.

FIG. 5 is a schematic drawing showing a second embodiment of a proximal tube (prior to formation of the hollow tube) formed of a sheet of a second material having a second Young's modulus and a plurality of strips of a first material having a first Young's modulus lower than the second Young's modulus.

FIG. 6 is a schematic drawing showing the proximal tube according to the embodiment of FIG. 5, after formation of the hollow tube.

FIG. 7 is a schematic drawing showing a third embodiment of a proximal tube (prior to formation of the hollow tube) formed of a plurality of sheets of a first material having a first Young's modulus and a plurality of strips of a second material having a second Young's modulus higher than the first Young's modulus, where each strip of the second material is inserted between sheets of the first material.

FIG. 8 is a schematic drawing showing a fourth embodiment of a proximal tube (prior to formation of the hollow tube) formed of a plurality of sheets of a second material having a second Young's modulus and a plurality of strips of a first material having a first Young's modulus lower than the second Young's modulus, where each strip of the first material is inserted between sheets of the second material.

FIG. 9 is a schematic drawing showing a proximal tube of a fifth embodiment in which the proximal tube is made of a hollow tube made of a first material having a first Young's modulus and having an area of decreased diameter, and a second material having a second Young's modulus higher than the first Young's modulus provided at an outer circumference of the area of decreased diameter.

FIG. 10 is a cross-section taken along line X-X of FIG. 9.

### DETAILED DESCRIPTION

Before turning to the figures, which illustrate the exemplary embodiments in detail, it should be understood that the present invention is not limited to the details or methodology set forth in the description or illustrated in the figures.

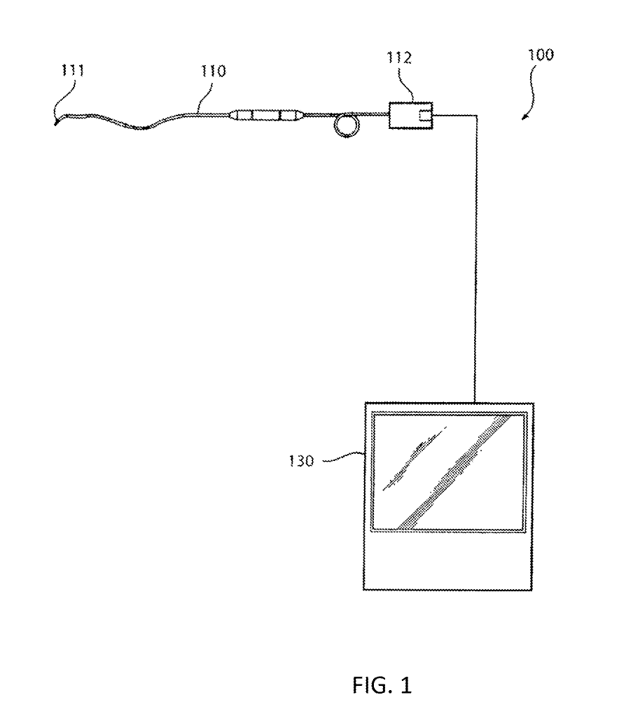

FIG. 1 shows a system 100 comprising a sensor guide wire according to one embodiment of the present invention. The arrangement comprises a sensor guide wire 110, and a physiological monitor 130. The sensor guide wire 110 may comprise a sensor element 111 arranged at the distal end of the sensor guide wire 110. The sensor element 111 may be arranged to sense a physiological or other variable in a living body, such as a human or animal body, and provide a sensor signal. The sensor guide wire 110 is a disposable device which typically includes a proximal connector 112 (which may be a female or male connector) for connection to the physiological monitor 130 which processes the sensor signal to generate a measurement of the physiological or other variable. Alternatively, a signal converting device or an interfacing device may be disposed between the proximal connector 112 and the physiological monitor 130, such as for example, the signal converting and interfacing devices disclosed in U.S. Patent Application Publication No. 2012/0289808, which is hereby incorporated by reference in its entirety for its teachings related to signal converting and interfacing devices, the use of physiological monitors, and the structure and use of sensor guide wire devices. Such a signal converting or interfacing device may be arranged to interface the sensor element 111 to the physiology monitor 130 such that a signal indicative of the physiological or other variable sensed by the sensor element 111 is pre-processed and forwarded to the physiology monitor 130. According to other embodiments, the sensor guide wire 110 can communicate via wireless transmission with the physiological monitor 130 such as, for example, the wireless transmission arrangement disclosed in U.S. Pat. Nos. 7,724,148; 8,174,395; and 8,461,997, which are hereby incorporated by reference in their entireties for their teachings related to wireless transmission arrangements between sensor guide wires and physiological monitors, and the structure and use of sensor guide wire devices.

The sensor element 111 may be used to sense any suitable physiological variable, such as, for example, pressure or temperature or flow. The sensor may be a microchip, a pressure sensitive device in the form of a membrane, a thermistor, a sensor for measuring the concentration or presence of a blood analyte, or other suitable pressure, temperature, or other variable-measuring device. Furthermore, the sensor element 111 may be a plurality of sensor devices. The physiological monitor 130 may use the sensor readings from the sensor element 111 to determine blood pressure, blood temperature, blood flow, the concentration or presence of one or more blood analytes, and/or Fractional Flow Reserve measurements (FFR). In short, FFR is used to identify constrictions of coronary vessels by obtaining the ratio between the pressure distally and proximally of a constriction.

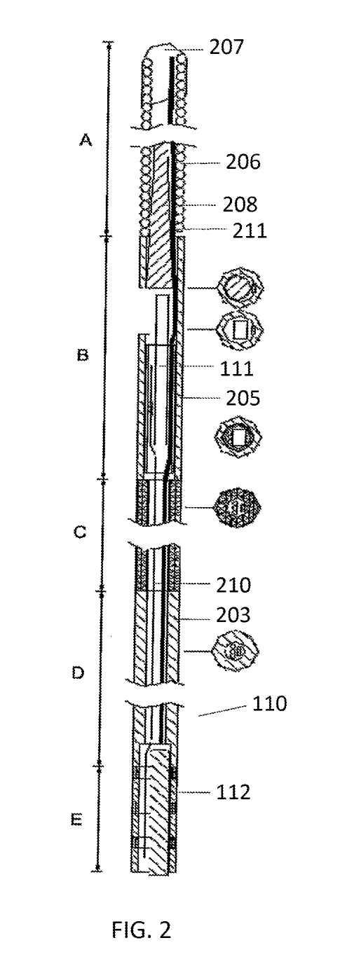

FIG. 2 shows a sensor guide wire 110 that can be used in the system of FIG. 1. The sensor guide wire 110 comprises the proximal connector 112, a flexible proximal tube 203, a jacket or sleeve 205, a distal end portion having a coil 206 and a tip 207, a distal core wire 208, and the sensor element 111, which is connected to the connector 112 by at least one electrical lead or microcable or optical signal line 210. The distal tip 207 may comprise an arced tip, which is connected to the core wire 208. The coil 206 may be a radioopaque coil made of, for example, platinum, but any suitable material (radioopaque or not) may be used. The coil 206 may be attached to the inner or outer circumference of the jacket 205 or the outer circumference of an enlarged portion of the core wire 208. In use, the connector 112 at the proximal end of the proximal tube 203 is inserted into a corresponding connector, such that measurement signals from the sensor element 111 can be displayed, for example as curves or numbers, on a suitable display on the physiology monitor 130.

The sensor element(s) 111 are connected to the microcables or optical signal lines 210, for transmitting signals between the sensor element 111 in the distal part of the guide wire and the connector 112 at the proximal end of the proximal tube 203. Examples of suitable microcables are described, for example, in U.S. Patent Application Publication No. 2010/0228112, U.S. Patent Application Publication No. 2011/0213220, and U.S. Patent Application Publication No. 2012/0289808, all of which are hereby incorporated by reference in their entireties for their teachings related to microcables in guide wire assemblies and the structure and use of guide wire assemblies.

The sensor guide wire 110 may optionally comprise a safety wire 211, which is attached in the tip 207 and extends preferably to the proximal connector 112. In case of an accidental break of the sensor guide wire 110 when, for example, a doctor tries to push the sensor guide wire 110 through a sharp bend in an artery of a patient, the safety wire 211 will make it possible to retrieve all parts of the sensor guide wire 110 from the patient's artery. The safety wire 211 may also be helpful during manufacturing of the sensor guide wire 110 in that the safety wire 211 can act as a guide when the different parts are assembled and threaded over each other. The safety wire 211 may alternatively have a shorter extension along the sensor guide wire 110, typically from the tip 207 to the jacket 205.

In FIG. 2, the capital letters A to D represent the length of the different sections of the sensor guide wire 110, and the following intervals should represent exemplifying lengths of the respective sections:

A=the length of the distal end portion=about 2 cm to about 3 cm;
B=the length of the jacket or sleeve 205=about 0.5 mm to about 10 mm, preferably about 1 mm to about 3 mm;
C=the length of a flexible portion (for example, a flexible tube, a braided tube, a flexible tubular braided wire or a flexible wire covered with a polyimide tube)=about 100 mm to about 500 mm;
D=the length of the proximal tube 203=about 135 cm to about 340 cm, preferably about 160 cm to about 300 cm;
E=the length of the proximal connector 112=about 10 mm to about 50 mm.

The diameter of the sensor guide wire 110 preferably varies between about 0.25 to about 2.5 mm; for use in coronary arteries, for example, the diameter is normally about 0.35 mm. In the context of length, width, diametrical, and other spatial dimensions, the modifier “about” can include a deviation of plus or minus 0 to 10% of the amount it modifies, preferably plus or minus 0 to 5% of the amount it modifies.

It should in particular be noted that the length of the jacket or sleeve 205 is rather small in comparison with the total length of the sensor guide wire. For example, the jacket or sleeve 205 can range about 0.01% to 5% of the total length of the sensor guide wire, preferably 0.025% to 2.5% of the total length of the sensor guide wire, more preferably 0.05% to 1.5% of the total length of the sensor guide wire.

The proximal tube 203 may be made of two or more materials, each having a different Young's modulus.

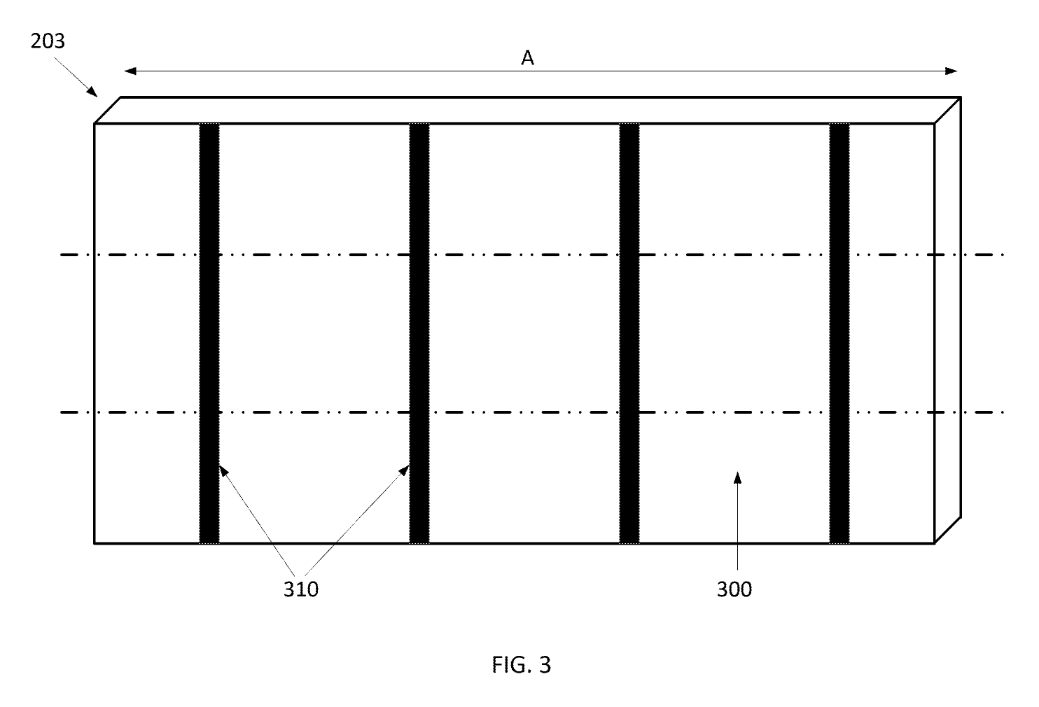

Referring now to FIGS. 3-10, the proximal tube 203 can be made of a first material 300 having a first Young's modulus and a second material 310 having a second Young's modulus that is higher than the first Young's modulus. The first Young's modulus of the first material 300 may be, for example, any Young's modulus equal to or less than 220 GPa. The second Young's modulus of the second material 310 may be, for example, any Young's modulus equal to or greater than 150 GPa. In the overlapping area between 150 GPa and 220 GPa, the first material 300 and the second material 310 may be selected such that the second Young's modulus is higher than the first Young's modulus.

The first material 300 may be stainless steel, a super elastic alloy, such as Nitinol, copper-tin, copper-zinc, or copper-zinc-tin, or another metal or metal alloy. The first material 300 may also be an Al—Mg—Cu alloy, an Al—Mg alloy, or an Al—Cu alloy. The second material 310 may be any material having a higher Young's modulus than the first material 300 selected. For example, the second material 310 may have a Young's modulus 10%, 30%, or 50% higher than the Young's modulus of the first material 300. For example, the second material 310 may be made of tungsten, molybdenum, alloys thereof, or another metal or metal alloy. In one example, if a very soft alloy such as a Cu alloy or an Al alloy having a low Young's modulus is used as the first material 300, steel can be used as the second material 310 since steel has a higher Young's modulus than a Cu alloy or an Al alloy.

The presence of the second material 310 (i.e., a high Young's modulus material) at or around the circumference of the proximal tube 203 improves the transfer of a rotational force from the proximal end to the distal end of the guide wire, when the guide wire is rotated at the proximal end. The presence of the first material 300 (i.e., a low Young's modulus material) at or around the circumference of the proximal tube 203 allows the guide wire to bend in a direction perpendicular to the longitudinal axis of the guide wire.

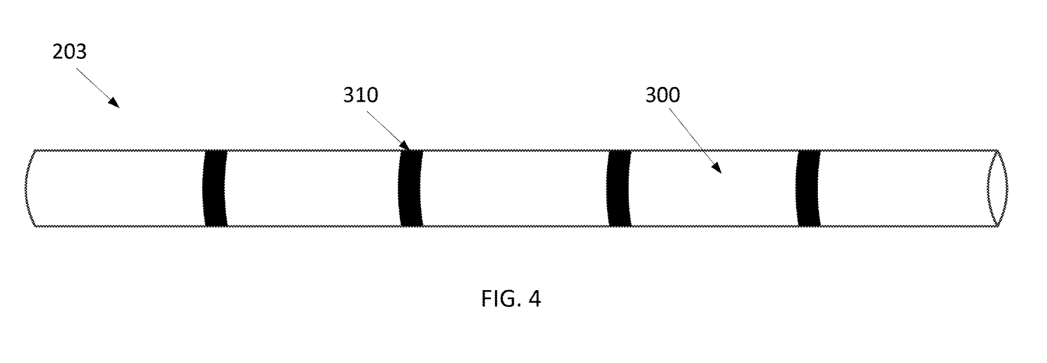

In the embodiment of FIGS. 3 and 4, to form the proximal tube 203, a plurality of strips of the second material 310 may be placed on a sheet of the first material 300 in intervals and at a 90 degree angle to a longitudinal direction A of the sheet of the first material 300. A width of each of the plurality of strips may be equal to 1 mm up to and including 10 cm. A thickness of the strip may be greater than or equal to 1 micrometer and less than a thickness of the proximal tube 203 (i.e., a distance between an inner diameter and an outer diameter of the proximal tube 203).

The first material 300 and the second material 310 are joined by roll bonding, the sheet of the first material 300 (with the second material 310) is rolled, and ends of the sheet are welded to form a hollow tube having varying stiffness along its length. In other words, the proximal tube 203 will have alternating segments of a low Young's modulus material (i.e., the first material 300) and a low Young's modulus material (i.e., the first material) reinforced with a high Young's modulus material (i.e., the second material 310). Other suitable methods of joining the first material 300 and the second material 310 may be used, for example, welding, a combination of welding and a known forming process, or an explosion welding/bonding technique. The plurality of strips of the second material 310 may be placed so that the second material 310 is provided on an outer circumference of the proximal tube 203 (see FIG. 4). Alternatively, the plurality of strips of the second material 310 may be placed so that the second material 310 is provided on an inner circumference of the proximal tube 203 (not illustrated). In either case, after joining the first material 300 and the second material 310, the plurality of strips of the second material 310 can be flush with the first material 300 such that the outer circumference and the inner circumference of the proximal tube 203 has a smooth surface and a consistent diameter.

The plurality of strips of the second material 310 may be evenly spaced along the full length of the proximal tube 203. The spacing of the plurality of strips of the second material 310 may be varied to adjust the flexibility and torqueability of the proximal tube 203. For example, the plurality of strips of the second material 310 may be spaced further apart in portions of the proximal tube 203 that require more flexibility (e.g., the end of the proximal tube 203 closer to the sensor element 111) and/or spaced closer together in portions of the proximal tube 203 that require more stiffness (i.e., less flexibility).

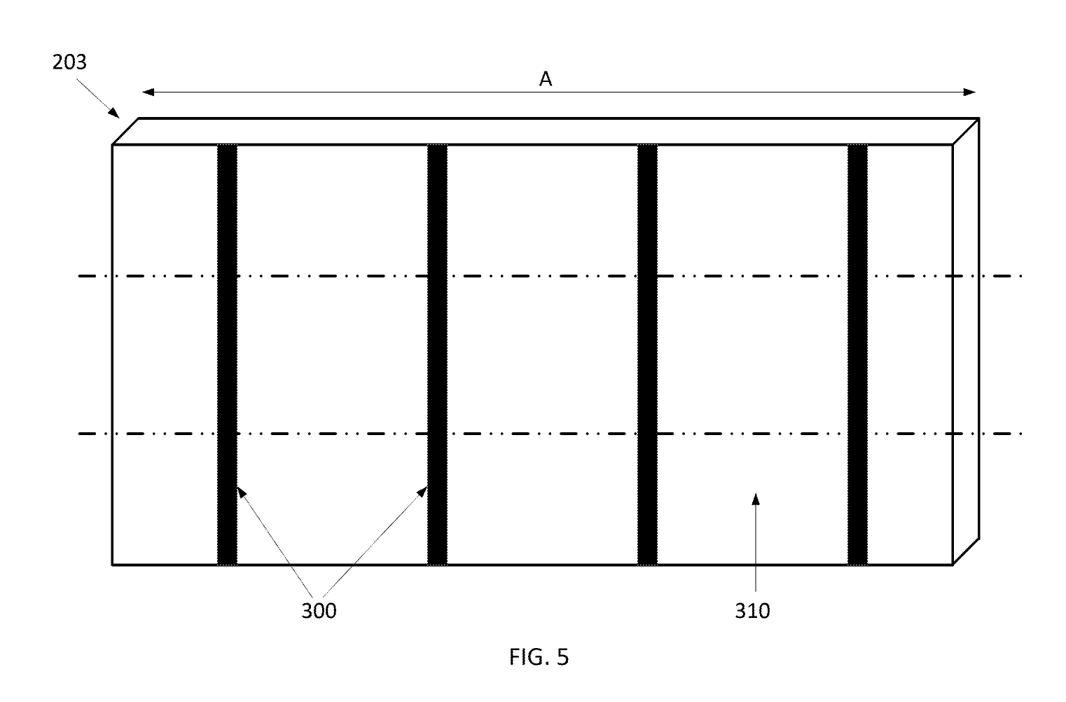

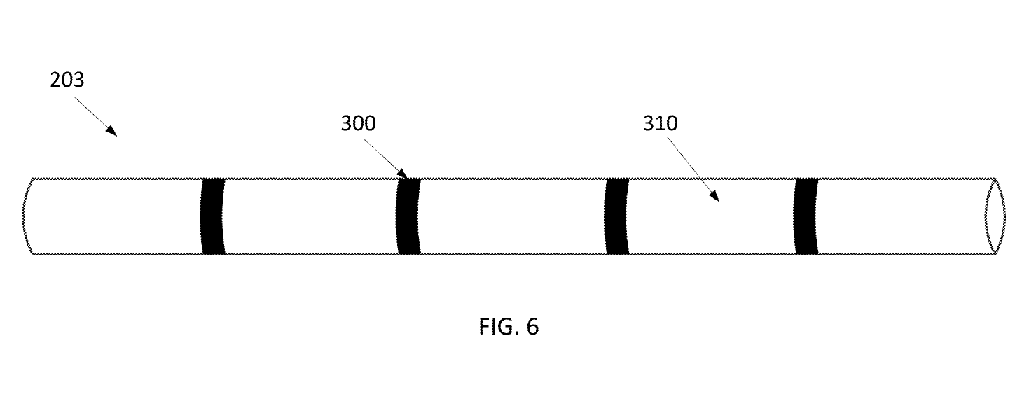

In the embodiment of FIGS. 5 and 6, to form the proximal tube 203, a plurality of strips of the first material 300 may be placed on a sheet of the second material 310 in intervals and at a 90 degree angle to a longitudinal direction A of the sheet of the second material 310. A width of each of the plurality of strips may be equal to 1 mm up to and including 10 cm. A thickness of the strip may be greater than or equal to 1 micrometer and less than a thickness of the proximal tube 203 (i.e., a distance between an inner diameter and an outer diameter of the proximal tube 203).

The first material 300 and the second material 310 are roll bonded, and the sheet of the second material 310 (with the first material 300) is rolled, and ends of the sheet are welded to form a hollow tube having varying stiffness along its length. In other words, the proximal tube 203 will have alternating segments of a high Young's modulus material (i.e., the second material 310) and a high Young's modulus material (i.e., the second material 310) reinforced with a low Young's modulus material (i.e., the first material 300). Other suitable methods of joining the first material 300 and the second material 310 may be used, for example, welding, a combination of welding and a known forming process, or an explosion welding/bonding technique. The plurality of strips of the first material 300 may be placed so that the first material 300 is provided on an outer circumference of the proximal tube 203 (see FIG. 6). Alternatively, the plurality of strips of the first material 300 may be placed so that the first material 300 is provided on an inner circumference of the proximal tube 203 (not illustrated). In either case, after joining the first material 300 and the second material 310, the plurality of strips of the first material 300 can be flush with the second material 310 such that the outer circumference and the inner circumference of the proximal tube 203 has a smooth surface and a consistent diameter.

The plurality of strips of the first material 300 may be evenly spaced along the full length of the proximal tube 203. The spacing of the plurality of strips of the first material 300 may be varied to adjust the flexibility and torqueability of the proximal tube 203. For example, the plurality of strips of the first material 300 may be spaced further apart in portions of the proximal tube 203 that require less flexibility (e.g., the end of the proximal tube 203 further from the sensor element 111) and/or spaced closer together in portions of the proximal tube 203 that require less stiffness (i.e., more flexibility).

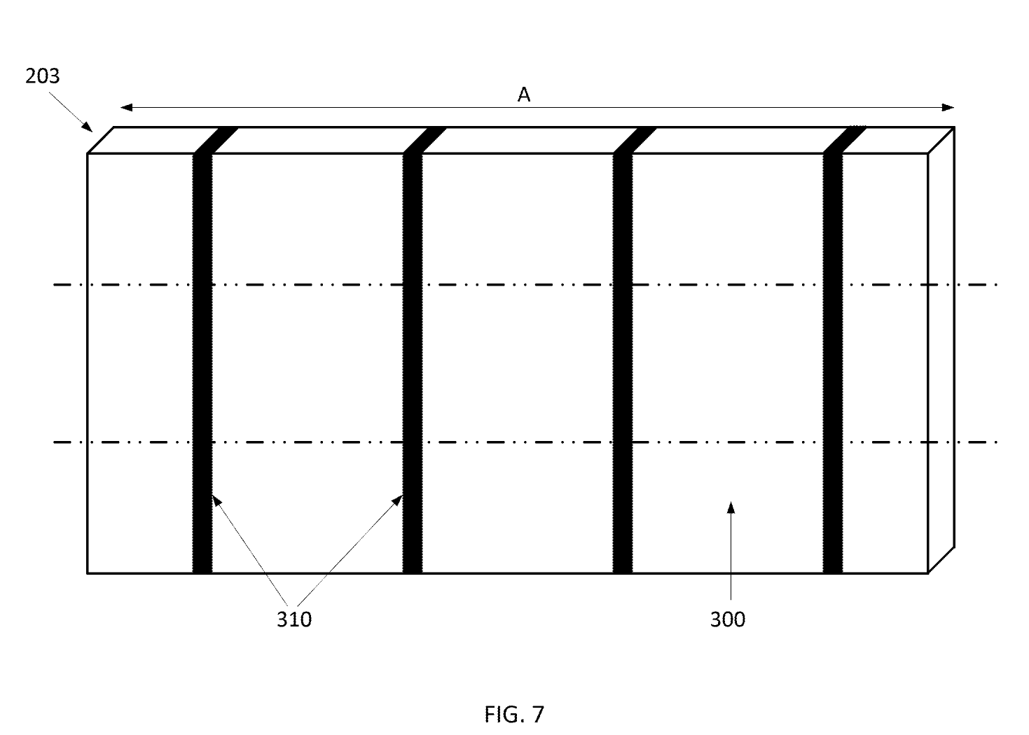

In the embodiment of FIG. 7, to form the proximal tube 203, a plurality of strips of the second material 310 may be inserted between sheets of the first material 300 in intervals and at a 90 degree angle to a longitudinal direction A of the proximal tube 203. A width of each of the plurality of strips may be equal to 1 mm up to and including 10 cm. A thickness of the strip is equal to a thickness of the proximal tube 203 (i.e., a distance between an inner diameter and an outer diameter of the proximal tube 203). After formation of the hollow tube, the proximal tube 203 has an outer appearance, as illustrated in FIG. 4. The plurality of strips of the second material 310 may be evenly spaced along the full length of the proximal tube 203. The spacing of the plurality of strips of the second material 310 may be varied to adjust the flexibility of the proximal tube 203. In another example, the plurality of strips of the second material 310 may be spaced further apart in portions of the proximal tube 203 that require more flexibility (e.g., the end of the proximal tube 203 closer to the sensor element 111) and/or spaced closer together in portions of the proximal tube 203 that require more stiffness (i.e., less flexibility).

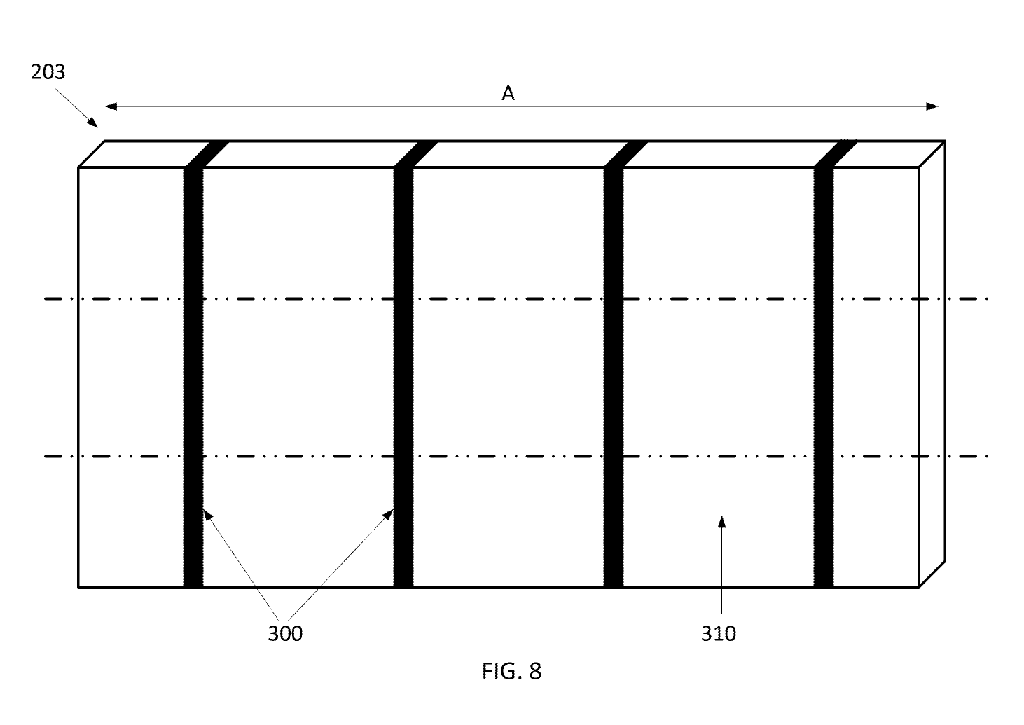

In the embodiment of FIG. 8, to form the proximal tube 203, a plurality of strips of the first material 300 may be inserted between sheets of the second material 310 in intervals and at a 90 degree angle to a longitudinal direction A of the proximal tube 203. A width of each of the plurality of strips may be equal to 1 mm up to and including 10 cm. A thickness of the strip is equal to a thickness of the proximal tube 203 (i.e., a distance between an inner diameter and an outer diameter of the proximal tube 203). After formation of the hollow tube, the proximal tube 203 has an outer appearance, as illustrated in FIG. 6. The plurality of strips of the first material 300 may be evenly spaced along the full length of the proximal tube 203. The spacing of the plurality of strips of the first material 300 may be varied to adjust the flexibility of the proximal tube 203. For example, the plurality of strips of the first material 300 may be spaced further apart in portions of the proximal tube 203 that require less flexibility (e.g., the end of the proximal tube 203 further from the sensor element 111) and/or spaced closer together in portions of the proximal tube 203 that require less stiffness (i.e., more flexibility).

In both of the embodiments of FIG. 7 and FIG. 8, the plurality of strips and the plurality of sheets are joined by roll bonding, rolled, and ends welded together to form a hollow tube having varying stiffness along its length. In other words, the proximal tube 203 will have alternating segments of a low Young's modulus material (i.e., the first material 300) and a high Young's modulus material (i.e., the second material 310). Other suitable methods of joining the first material 300 and the second material 310 may be used, for example, welding, a combination of welding and a known forming process, or an explosion welding/bonding technique. After joining the first material 300 and the second material 310, the plurality of strips will be flush with the plurality of sheets such that the outer circumference and the inner circumference of the proximal tube 203 has a smooth surface and a consistent diameter.

In the embodiments illustrated in FIGS. 3-8, a dimension of each strip of one material extending along the longitudinal direction A is smaller than a dimension of each-sheet of the other material extending along the longitudinal direction A. Alternatively, the dimension of each strip of one material may be equal to the dimension of each sheet of the other material. In addition, the strips of material may be provided at angles other than 90 degrees with respect to the longitudinal axis of the guide wire.

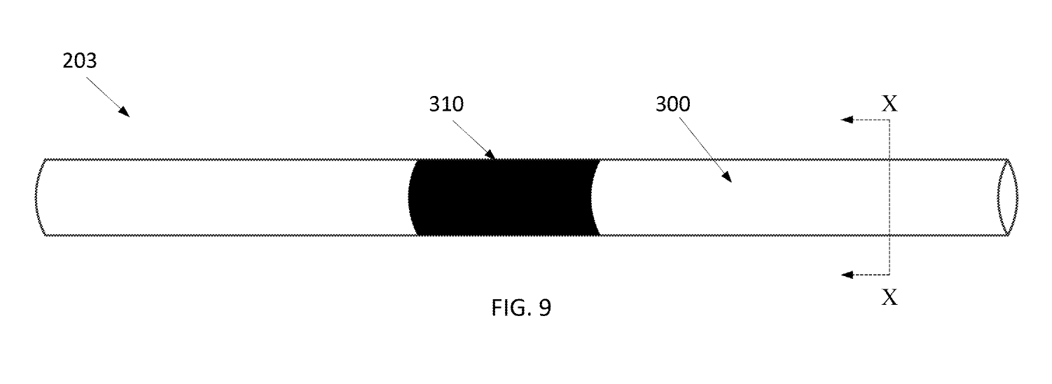

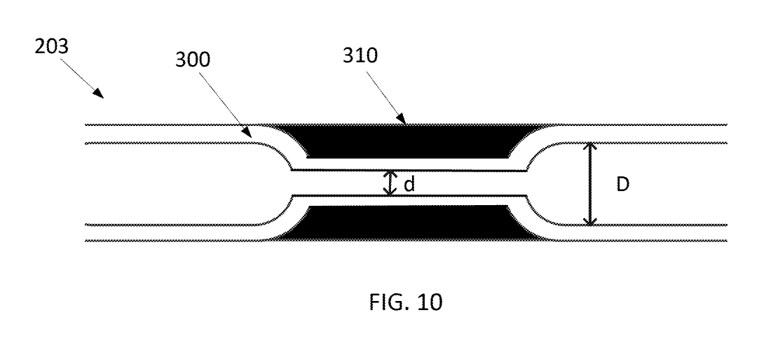

Referring now to FIGS. 9 and 10, in another embodiment, a continuous tube made from the first material 300 may be provided with areas of decreased diameter d (compared to a diameter D) along the length of the tube. On an outer circumference of the tube, in the areas of decreased diameter d, strips of the second material 310 may be roll bonded, welded or otherwise joined to the first material 300 to form the proximal tube 203. After the first material 300 and the second material 310 are joined together, the strips of the second material 310 can be flush with the first material 300 such that the outer circumference of the proximal tube 203 has a smooth surface and a consistent diameter.

In the examples discussed above, two materials are utilized to form the proximal tube 203. However, it should be understood that three or more materials may be used, provided that at least two of the materials have a different Young's modulus.

In the examples discussed above, each of the plurality of strips has a same width. However, it should be understood that strips of varying widths may be used along a length of the proximal tube.

It should be understood that even more variations of proximal tube shapes and opening alignments are possible. For example, the proximal tube 203 may include any number of cylindrical sections of different diameters, and may include any number of tapered sections or other sections to transition between the cylindrical sections, provided that the tube is mainly formed from the first material 300 and the cylindrical sections of decreased diameter or tapered sections are reinforced with the second material 310. The cross-section of the proximal tube 203 may be of any shape (e.g., rectangular, ovoid, spherical, etc.).

The proximal tube described in any of the embodiments may be used in place of the jacket 205 or other parts of a guide wire. In this case, distal openings and side openings placed through the wide wall of the proximal tube 203 may be located in any of the sections of the proximal tube 203 such that fluids flow into the guide wire at a distal end of the guide wire through the distal openings, and flows over the sensor element 111 (e.g., a pressure sensor) and through the side openings (or vice versa). The proximal tube described in any of the embodiments may be used with a guide wire without a core wire. The proximal tube described in any of the embodiments may also be used with a guide wire without a sensor or a guide wire with a braided portion.

The construction and arrangements of the sensor guide wire, as shown in the various exemplary embodiments, are illustrative only. Although only a few embodiments have been described in detail in this disclosure, many modifications are possible (e.g., variations in sizes, dimensions, structures, shapes and proportions of the various elements, values of parameters, mounting arrangements, use of materials, orientations, etc.) without materially departing from the novel teachings and advantages of the subject matter described herein. Some elements shown as integrally formed may be constructed of multiple parts or elements, the position of elements may be reversed or otherwise varied, and the nature or number of discrete elements or positions may be altered or varied. Other substitutions, modifications, changes and omissions may also be made in the design, operating conditions and arrangement of the various exemplary embodiments without departing from the scope of the present invention. Features of one embodiment may be combined with a feature of another embodiment.

As utilized herein, the terms “approximately,” “about,” “substantially”, and similar terms are intended to have a broad meaning in harmony with the common and accepted usage by those of ordinary skill in the art to which the subject matter of this disclosure pertains. It should be understood by those of skill in the art who review this disclosure that these terms are intended to allow a description of certain features described and claimed without restricting the scope of these features to the form provided. Accordingly, these terms should be interpreted as indicating that insubstantial or inconsequential modifications or alterations of the subject matter described and claimed are considered to be within the scope of the invention.

What is claimed is:

1. A sensor guide wire for an intravascular measurement of a physiological variable in a living body, comprising:
a sensor element configured to measure the physiological variable; and
a proximal tube comprising a first material having a first Young's modulus and a second material having a second Young's modulus,
wherein the second Young's modulus is higher than the first Young's modulus, and the second material is configured to improve torqueability.

2. The sensor guide wire of claim 1, wherein the first material is selected from the group consisting of stainless steel and super elastic alloy.

3. The sensor guide wire of claim 2, wherein the super elastic alloy is selected from the group consisting of Nitinol, copper-tin, copper-zinc, and copper-zinc-tin.

4. The sensor guide wire of claim 1, wherein the second material is comprised of tungsten, molybdenum, or alloys thereof.

5. The sensor guide wire of claim 1, wherein
the proximal tube comprises
a hollow tube made of one of the first material and the second material; and
a plurality of strips made of the other of the first material and the second material provided on the hollow tube, and
the plurality of strips are spaced along a length of the hollow tube.

6. The sensor guide wire of claim 5, wherein at least some of the plurality of strips are evenly spaced along the length of the hollow tube.

7. The sensor guide wire of claim 5, wherein at least some of the plurality of strips are unevenly spaced along the length of the hollow tube.

8. The sensor guide wire of claim 5, wherein the plurality of strips are provided on an inner circumference of the hollow tube.

9. The sensor guide wire of claim 5, wherein the plurality of strips are provided on an outer circumference of the hollow tube.

10. The sensor guide wire of claim 1, wherein
the proximal tube comprises a hollow tube made of sheets of one of the first material and the second material and a plurality of strips made of the other of the first material and the second material, the plurality of strips inserted between the sheets, and
the plurality of strips are spaced along a length of the hollow tube.

11. The sensor guide wire of claim 10, wherein at least some of the plurality of strips are evenly spaced along the length of the hollow tube.

12. The sensor guide wire of claim 10, wherein at least some of the plurality of strips are unevenly spaced along the length of the hollow tube.

13. The sensor guide wire of claim 10, wherein the plurality of strips comprise portions of an inner circumference of the hollow tube.

14. The sensor guide wire of claim 10, wherein the plurality of strips comprise portions of an outer circumference of the hollow tube.

15. The sensor guide wire of claim 10, wherein the hollow tube has a constant diameter.

16. The sensor guide wire of claim 1, wherein
the proximal tube comprises a hollow tube made of the first material, the hollow tube including at least one area of decreased diameter of the first material along a length thereof, and
the second material is provided at an outer circumference of the at least one area of decreased diameter.

17. The sensor guide wire of claim 1, wherein the guide wire has no core wire.

18. A method of forming a proximal tube for a sensor guide wire for an intravascular measurement of a physiological variable in a living body, the sensor guide wire comprising a sensor element configured to measure the physiological variable, the method comprising:
providing at least one sheet made of one of 1) a first material having a first Young's modulus or 2) a second material having a second Young's modulus, the second Young's modulus being higher than the first Young's modulus;
providing a plurality of strips made of the other of the first material and the second material;
joining the first material and the second material; and
forming the joined first material and the second material into a hollow tube having the plurality of strips spaced along a length of the hollow tube.

19. The method of claim 18, wherein a sheet is provided and the plurality of strips are placed on the sheet prior to bonding the first material and the second material such that reinforced portions of the hollow tube include the first material having the first Young's modulus and the second material having the second Young's modulus.

20. The method of claim 18, wherein a plurality of sheets is provided and each strip is inserted between two sheets prior to joining the first material and the second material such that the hollow tube comprises alternating segments of the first material having the first Young's modulus and the second material having the second Young's modulus.

21. The method of claim 18, wherein at least some of the plurality of strips are evenly spaced along the length of the hollow tube.

22. The method of claim 18, wherein at least some of the plurality of strips are unevenly spaced along the length of the hollow tube.

23. The method of claim 18, wherein
the at least one sheet is made of the first material;
the plurality of strips are made of the second material;
forming the joined first material and the second material comprises forming a tube having at least one area of decreased diameter along a length thereof; and
at least one strip from the plurality of strips is provided in an area of decreased diameter.

24. A guide wire comprising:
a tubular member having along its length a first material having a first Young's modulus and a second material having a second Young's modulus,
wherein the second Young's modulus is higher than the first Young's modulus, and
wherein the tubular member has alternating portions of the first material and the second material.

25. The guide wire of claim 24, wherein the tubular member comprises alternating portions of the first material and portions of the first material reinforced with the second material.

26. The guide wire of claim 24, wherein the tubular member comprises alternating portions of the second material and portions of the second material reinforced with the first material.
